(19) United States
(12) Patent Application Publication
(10) Pub. No.: US 2023/0014454 A1
(43) Pub. Date: Jan. 19, 2023

(54) FORMING A CUSTOM FITTED MESH BASED ON A TOPOGRAPHICAL MAP OF A PATIENT'S ABDOMINAL CAVITY

(71) Applicant: Jorge L. Florin, Windermere, FL (US)

(72) Inventor: Jorge L. Florin, Windermere, FL (US)

(21) Appl. No.: 17/863,479

(22) Filed: Jul. 13, 2022

Related U.S. Application Data
(60) Provisional application No. 63/221,063, filed on Jul. 13, 2021.

(51) Int. Cl. B29C 64/386; A61B 34/10; A61B 34/30; A61B 90/00; B33Y 50/00; G06F 30/17

(52) U.S. Cl. CPC B29C 64/386; A61B 34/10; A61B 34/30; A61B 90/37; B33Y 50/00; G06F 30/17; A61B 2034/105; A61B 2034/302; B33Y 80/00

(57) ABSTRACT

The present disclosure relates generally to hernia repair, and more specifically to forming a custom fitted mesh based on a topographical map of at least one portion of a patient's abdominal cavity. Some specific aspects of the present disclosure relate to exemplary methods, systems, devices and computer readable mediums for forming a custom fitted mesh based on a topographical map of the at least one portion of the patient's abdominal cavity.

### RELATED APPLICATIONS

This application claims the benefit of pending U.S. Provisional Application No. 63/221,063, filed Jul. 13, 2021, which is incorporated herein by reference in its entirety for all purposes.

### FIELD

The present disclosure relates generally to hernia repair, and more specifically to forming a custom fitted mesh based on a topographical map of at least one portion of a patient's abdominal cavity.

### BACKGROUND

Meshes may be implanted into a patient to heal or form a scaffold for healing hernia defects. However, current hernia meshes are standardized and thus not custom fitted to a patient. Instead, standard meshes must be modified during a surgery. Sometimes, a surgeon will need to manually bend the mesh to fit the contours of the at least one portion of the patient's abdominal cavity, which can be inefficient, especially when the surgery is performed laparoscopically or robotically. In addition, even when the mesh is manually bent to fit the patient, the mesh may nevertheless fit poorly, leading to complications. Accordingly, methods, systems, devices, and computer readable mediums for forming custom fitted meshes are needed.

### SUMMARY

A method for forming a custom fitted mesh based on a topographical map of at least one portion of a patient's abdominal cavity is described. One or more embodiments of the method include allowing at least one user to obtain, using at least one camera of at least one device, a real-time representation of at least one portion of a patient's abdominal cavity, displaying the real-time representation of the at least one portion of the patient's abdominal cavity on at least one screen, allowing the at least one user of the least one device to create a topographical map of a contour of the at least one portion of the patient's abdominal cavity, and instructing at least one machine to form a custom fitted mesh based on the topographical map, where the custom fitted mesh conforms to the contour of the at least one portion of the patient's abdominal cavity.

A first exemplary system for forming a custom fitted mesh based on a topographical map of at least one portion of a patient's abdominal cavity is described. One or more embodiments of the system include at least one device comprising at least one screen, where the at least one screen is configured to display a real-time representation of the at least one portion of the patient's abdominal cavity, at least one camera, where the at least one camera is configured to obtain a real-time representation of the at least one portion of the patient's abdominal cavity and allow at least one user of the at least one device to create a topographical map of the at least one portion of the patient's abdominal cavity from the real-time representation, and at least one machine, where the at least one machine is configured to form a custom fitted mesh based on the topographical map of the at least one portion of the patient's abdominal cavity.

A second exemplary system for forming a custom fitted mesh based on a topographical map of at least one portion of a patient's abdominal cavity is described. One or more embodiments of the system include at least one surgical robot comprising at least one arm, where the at least one arm comprises at least one surgical camera configured to obtain a real-time representation of the at least one portion of the patient's abdominal cavity, at least one screen, where the at least one screen is configured to display the real-time representation of the at least one portion of the patient's abdominal cavity, and at least one three-dimensional (3D) printer, where the at least one 3D printer is configured to form a custom fitted mesh based on the topographical map of the at least one portion of the patient's abdominal cavity.

A device for forming a custom fitted mesh based on a topographical map of at least one portion of a patient's abdominal cavity are described. One or more embodiments of the device include at least one screen, where the at least one screen is configured to display a real-time representation of the at least one portion of the patient's abdominal cavity and at least one camera, where the at least one camera is configured to obtain a real-time representation of the at least one portion of the patient's abdominal cavity and allow at least one user of the at least one device to create a topographical map of the at least one portion of the patient's abdominal cavity from the real-time representation, where the device is configured to instruct at least one machine to form a custom fitted mesh based on the topographical map of the at least one portion of the patient's abdominal cavity.

A non-transitory computer readable medium for forming a custom fitted mesh based on a topographical map of at least one portion of a patient's abdominal cavity are described. One or more embodiments of the non-transitory computer readable medium include code comprising instructions executable by a processor to receive, from at least one camera of at least one device, data corresponding to a real-time representation of the at least one portion of the patient's abdominal cavity, instruct at least one screen to display the real-time representation of the at least one portion of the patient's abdominal cavity, receive instructions from at least one user of the least one device to create a topographical map of a contour of the at least one portion of the patient's abdominal cavity, and instruct at least one machine to form a custom fitted mesh based on the topographical map of the at least one portion of the patient's abdominal cavity, where the custom fitted mesh conforms to the contour of the at least one portion of the patient's abdominal cavity.

### BRIEF DESCRIPTION OF THE DRAWINGS

FIGS. 1 through 3 show examples of a system for forming a custom fitted mesh based on a topographical map of at least one portion of a patient's abdominal cavity according to aspects of the present disclosure.

FIG. 4 shows an example of a real-time representation of at least one portion of a patient's abdominal cavity, as displayed on a screen according to aspects of the present disclosure.

FIG. 5 shows an example of a topographical map overlayed on a real-time representation of at least one portion of a patient's abdominal cavity, as displayed on a screen according to aspects of the present disclosure.

FIG. 6 shows an example of a three-dimensional (3D) printer used to form a custom fitted mesh according to aspects of the present disclosure.

FIG. 7 shows an example of a process for forming a custom fitted mesh based on a topographical map of at least one portion of a patient's abdominal cavity according to aspects of the present disclosure.

### DETAILED DESCRIPTION

A method, system, device, and non-transitory computer readable medium for forming a custom fitted mesh based on a topographical map of a patient's abdominal cavity is described by some embodiments of the present disclosure. As used herein, a “custom fitted mesh” is a mesh that conforms to a contour of a patient's abdominal cavity. In certain examples, the custom fitted mesh conforms to a contour of at least one portion of the patient's abdominal cavity. In certain examples, the custom fitted mesh conforms to a contour of at least one portion of a patient's abdominal cavity omitting the at least one hernia defect from the contour of the custom fitted mesh, in some examples, allowing the custom fitted mesh to act a scaffold for healing the at least one hernia defect. In certain examples, the custom fitted mesh conforms to the contour of a least a portion of the patient's abdominal cavity that represents at least one anchor point for the custom fitted mesh against the at least one portion of the patient's abdominal cavity. In certain examples, the custom fitted mesh conforms to a contour of an entirety of the patient's abdominal cavity. In certain examples the custom fitted mesh conforms to a contour of the entirety of the patient's abdominal cavity omitting the contour of at least one hernia defect from the contour of the custom fitted mesh, in some examples, allow the custom fitted mesh to act a scaffold for healing the at least one hernia defect. While the specifics of the at least one portion of the patient's abdominal cavity are not particularly limited, in some implementations, the at least one portion of the patient's abdominal cavity comprises the patient's myopectineal orifice and pelvic floor. In some examples, the at least one portion of the patient's abdominal cavity comprises at least one abdominal wall (e.g., the anterior abdominal wall, myopectineal orifice, pelvic floor, any other portion of the walls or fascia forming the patient's abdominal cavity) or one portion of at least one abdominal wall. In further implementations, the at least one portion of the patient's abdominal cavity comprises the patient's diaphragm. In further implementations, the at least one portion of the patient's abdominal cavity is the entirety of the patient's abdominal cavity. In some such examples, the custom fitted mesh conforms to a contour of the entirety of the patient's abdominal cavity.

One or more embodiments of the method, system, device, and computer readable medium include allowing at least one user to obtain, using at least one camera of at least one device, a real-time representation of at least one portion of a patient's abdominal cavity. As used herein, a “real-time representation of at least one portion of a patient's abdominal cavity” is a still image, a moving image, or other visual representation that depicts at least one portion of a patient's abdominal cavity in “real time.” As used herein, the term “real-time” is directed to an event/action that can occur instantaneously or almost instantaneously in time when another event/action has occurred. In some embodiments, the terms “instantaneous,” “instantaneously,” “instantly,” and “in real time” refer to a condition where a time difference between a first time when an image is captured and a second time when the image is displayed is no more than 1 second. In some embodiments, the time difference between capture and display is between less than 1 second and several seconds. In some examples, the real-time representation of at least one portion of a patient's abdominal cavity comprises a real time representation of the patient's myopectineal orifice and pelvic floor. In some examples, the real-time representation comprises a real time representation of another portion of the patient's abdominal cavity.

In some examples, the patient is a mammal. In some examples, the mammal is a human. In some examples, the at least one portion of the patient's abdominal cavity was previously dissected. In some examples, the at least one portion of the patient's abdominal cavity was previously dissected during a surgical procedure.

In some examples, the at least one device is at least one surgical robot. Non-limiting examples of the at least one surgical robot include: single and multi-port robots manufactured by Intuitive Surgical™ (e.g., the DaVinci™); and the Hugo™ from Medtronic™. In some examples, the at least one device may comprise at least one computing device as described in further detail below.

In some examples, the at least one camera is at least one surgical camera. In some examples, the at least one surgical camera comprises a laparoscopic camera, which may or may not be present on the at least one surgical robot. In some implementations, the at least one surgical camera comprises a robotic camera, where the robotic camera is present on the at least one surgical robot. Some examples of the method, device, non-transitory computer readable medium, and system further include inserting the at least one surgical camera into the patient using the at least one surgical robot. Some examples further include navigating the at least one surgical camera toward the at least one portion of the patient's abdominal cavity. Some examples further include capturing, using the at least one surgical camera, the real time representation of the at least one portion of the patient's abdominal cavity. In some examples, the at least one camera may comprise an imaging device, such as, but not limited to an optical imaging device, a light sensing optical device, an infrared or near infrared imaging device, an ultrasonic imaging device, an x-ray device, an electromagnetic imaging device, or a magnetic resonance imaging (MRI) device.

Some examples further include displaying the real-time representation of the at least one portion of the patient's abdominal cavity on at least one screen. In some examples, the at least one screen is part of the at least one device (e.g., at least one surgical robot). In some examples, the at least one screen is at least one external screen. In some examples, the at least one screen is present on a computer, a mobile device, a virtual reality (VR) or augmented reality (AR) device, as a projected image (e.g., as a hologram), or any combination thereof. In some examples, the at least one screen is an eyepiece, a light-emitting-diode (LED) display, a liquid-crystal-display (LCD), or any other suitable screen. In some examples, the topographical map is virtually overlaid over the real-time representation of the at least one portion of the patient's abdominal cavity on the at least one screen.

Some implementations of the method further include allowing the at least one user of the least one device to create a topographical map of a contour of the at least one portion of the patient's abdominal cavity. As used herein, a “topographical map” is a virtual geometric shape or structure (e.g., a vector space or vector field) that conforms to a contour of the at least one portion of the patient's abdominal cavity. In some examples, the at least one user comprises at least one medical professional. In some implementations the at least one medical professional comprises a doctor, a nurse, a technician, a surgeon, or any combination thereof. In some such implementations, the at least one medical professional may manually guide at least one camera to different points of the at least one portion of the patient's abdominal cavity. In certain implementations, the manual guiding of at least one camera is performed with assistance of least one surgical robot comprising at least one surgical camera. In some implementations, the at least one user comprises the at least one surgical robot. In some such implementations, the at least one surgical robot may automatically (e.g., based on instructions from a computer readable medium) direct at least one surgical camera to topographically map a contour of the at least one portion of the patient's abdominal cavity. In some examples, the contour of the at least one portion of the patient's abdominal cavity is a contour of the patient's myopectineal orifice and pelvic floor. In some examples, the contour of the at least one portion of the patient's abdominal cavity is a contour of another portion of the patient's abdominal cavity. In certain examples device allows a user to create a topographical map that conforms to a contour of at least one portion of a patient's abdominal cavity omitting the contour of at least one hernia defect from the topographical map. In such examples, the topographical map created represents the at least one portion of the patient's abdominal cavity as it should appear after repair of the at least one hernia defect.

The topographical map may be a vector map in certain implementations. As used herein, a “vector map” is a geometric structure spanned by a set of vectors. Some examples of the method, device, non-transitory computer readable medium, and system further include constructing the vector map. In some examples, constructing the vector map comprises defining a fixed point on the real-time representation of at least one portion of a patient's abdominal cavity. The location of the fixed point is not necessarily limited, so long as the fixed point can be used to construct the vector map in applicable embodiments. For instance, in some non-limiting examples, the fixed point may correspond a center of mass of the at least one portion of the patient's abdominal cavity. In some non-limiting examples, the fixed point may correspond to a center of mass of the patient's myopectineal orifice and pelvic floor. In some non-limiting examples, the fixed point may be arbitrary. Some examples of constructing a vector map may further include defining a plurality of end points on the real-time representation of at least one portion of a patient's abdominal cavity. These endpoints, may in some implementations, correspond to contoured regions of the at least one portion of the patient's abdominal cavity. In some examples, the contoured regions may be identified and measured manually by the at least one user (e.g., by guiding at least one surgical camera to each contoured region and recording the measurement using the at least one surgical robot or by guiding at least one laparoscopic camera to each contoured region). In some examples, the contoured regions may be identified and measured automatically (e.g., based on instructions from a computer readable medium or by using a machine learning algorithm). Some examples of constructing the vector map may further include defining a plurality of vectors, where each of the plurality of vectors starts at the fixed point and terminates at one of the end points. In some such examples, the defining a plurality of vectors may be performed by measuring a distance between the fixed point and each of the end points. Some examples further include calculating the vector map, where the vector map is calculated by determining a vector space defined by the plurality of vectors. In some examples, the vector space is determined by calculating a cross product of the plurality of vectors. In some examples, the vector space is determined by calculating a vector field comprising the plurality of vectors. In certain implementations, a gradient of the vector field may conform to the contour of the at least one portion of the patient's abdominal cavity, including, but not limited to, a contour of the patient's myopectineal orifice and pelvic floor. In certain examples the vector map conforms to a contour of at least one portion of a patient's abdominal cavity omitting the contours of at least one hernia defect from the vector map. In such examples, the vector map created represents the at least one portion of the patient's abdominal cavity as it should appear after repair of the at least one hernia defect.

Some examples of the method, device, non-transitory computer readable medium, and system further include displaying the topographical map (e.g., vector map) on the at least one screen. In some examples, the at least one screen is present on the at least one device (e.g., the at least one surgical robot). Some examples of the method, device, non-transitory computer readable medium, and system include displaying the topographical map (e.g., vector map) on at least one external screen (i.e., external to the at least one device). In some examples, the external screen may be present on the at least one machine (e.g., on at least one three-dimensional (3D) printer or press machine). In certain implementations where the at least one screen comprises an eyepiece, the displaying of the topographical map on the at least one screen may comprise viewing the topographical map through the eyepiece.

Some examples of the method, device, non-transitory computer readable medium, and system further include allowing the at least one user to determine a location and dimensions of at least one hernia defect or a plurality of hernia defects. Some examples further include allowing the at least one user to modify the topographical map of the at least one portion of the patient's abdominal cavity to omit the at least one hernia defect or the plurality of hernia defects. Omitting the at least one hernia defect from the topographical map may, in some examples, allow a resulting custom fitted mesh to act a scaffold for healing the at least one hernia defect.

In some examples, the topographical map may be formed from a pre-set topographical template. Accordingly, some examples of the method, system, device, and non-transitory computer readable medium further include selecting a pre-set topographical template using the at least one screen. Some examples further include overlaying, using the at least one screen, the pre-set topographical template over the real-time representation of the at least one portion of the patient's abdominal cavity. Some examples further include warping the pre-set topographical template to match the contour of the at least one portion of the patient's abdominal cavity. In some examples, the warping is performed using the at least one screen. In some examples, the warping is performed by the at least one user. In some examples, the warping is performed automatically (e.g., based on instructions from a computer readable medium or by using a machine learning algorithm). In some examples, the warping is performed using the at least one camera. In certain examples the warping of the pre-set topographical template to match the contour of the at least one portion of the patient's abdominal cavity omits, skips, or leaves unwarped at least one portion of the pre-set topographical template corresponding to the at least one hernia defect. In such examples, the resulting topographical map created represents the pre-set topographical template warped to match at least one portion of the patient's abdominal cavity as it should appear after repair of the at least one hernia defect.

Additional exemplary methods of forming topographical maps, including vector maps may be found in US Patent Application Publication 2018/0116519 and US Patent Application Publication 2019/0082779 each of which is incorporated by reference herein in its respective entirety.

Certain aspects of the present method, system, device, and non-transitory computer readable medium include instructing at least one machine to form a custom fitted mesh that conforms to the contour of the at least one portion of the patient's abdominal cavity. In certain implementations, the at least one machine is instructed (e.g., over at least one computer network) by at least one device described herein to form the custom fitted mesh based on the topographical map. In certain implementations, instructions for the at least one machine to form the custom fitted mesh based on the topographical map are saved onto a suitable computer readable medium (as described herein). In some such embodiments, the saved instructions can be downloaded to the at least one machine, which would then form the custom fitted mesh based on the saved instructions.

In some examples, the at least one machine comprises at least one 3D printer. Examples of at least one 3D printer that may be suitable for certain aspects of the present disclosure include but are not limited to the 3D printers described in: US Patent Application Publication 2017/0165908 and US Patent Application Publication 2018/0049858, each of which is incorporated by reference herein in its respective entirety. Some examples of the method, system, device, and non-transitory computer readable medium include 3D printing the custom fitted mesh based on the topographical map. In certain implementations, the 3D printer is instructed by the at least one surgical robot to form the custom fitted mesh based on the topographical map. In certain implementations, instructions for the at least one 3D printer to form the custom fitted mesh based on the topographical map are saved onto a suitable computer readable medium (as described herein). In some such embodiments, the saved instructions can be downloaded to the at least one 3D printer, which would then form the custom fitted mesh based on the saved instructions.

In some examples, the at least one machine comprises a press machine. The specific type of press machine is not particularly limited and may include: a heat press, a vacuum press, a magnetic press, a hydraulic press, a push press, or any combination thereof. Specific on-limiting examples of suitable press machines are described in: US Patent Application Publication 2016/0346424 and US Patent Application Publication 2015/0297798, each of which is incorporated by reference herein in its respective entirety.

In certain implementations, the press machine is instructed by the at least one surgical robot to form the custom fitted mesh based on the topographical map. In certain implementations, instructions for the at least one press machine to form the custom fitted mesh based on the topographical map are saved onto a suitable computer readable medium (as described herein). In some such embodiments, the saved instructions can be downloaded to the at least one press machine, which would then form the custom fitted mesh (e.g., by pressing a standardized mesh as described below) based on the saved instructions.

Some examples of the method, system, device, and non-transitory computer readable medium may further include obtaining a standardized mesh and pressing, based on the topographical map, the standardized mesh into the custom fitted mesh. As used herein, a “standardized mesh” is a mesh that is sized, both in area and opening size, to fit at least one portion of an abdominal cavity of at least one mammal, including at least one human.

In some examples, the standardized mesh or custom fitted mesh may comprise at least one of: polycaprolactone, polypropylene, polyglycolic acid, polytetrafluoroethylene (PTFE), polyglactin, poliglecaprone, cellulose, or a combination thereof. In some examples, the standardized mesh or custom fitted mesh may comprise at least one material that maintains shape during surgery (i.e., a “shape memory material.”) Non-limiting examples of suitable shape memory materials are described in US Patent Application Publication 2009/0118747 and US Patent Application Publication 2011/0282365 each of which is incorporated by reference herein in its respective entirety. The shape of the standardized mesh or custom fitted mesh is not particularly limited and may: be symmetrical, be asymmetrical, have a rectangular cross-section, have a circular cross-section, have an elliptical cross-section, have a triangular cross section, or any combination thereof. Opening sizes of the standardized mesh or custom fitted mesh may be uniform or non-uniform, without limitation. In some examples, the standardized mesh or custom fitted mesh may have a specific average opening size, where “the average opening size” is an averages size of at least one dimension (e.g., length, width, radius, diameter) measured across all openings of the standardized mesh or custom fitted mesh. In certain examples, the average opening size may range from: 0.01 mm to 10 mm, 0.1 mm to 10 mm, 1 mm to 10 mm, 5 mm to 10 mm, 0.01 mm to 5 mm, 0.01 mm to 1 mm, 0.01 mm to 0.1 mm, 0.1 mm to 1 mm, 0.1 mm to 5 mm, 1 mm to 5 mm, 2 mm to 9 mm, 3 mm to 8 mm, 4 mm to 7 mm, or 5 mm to 6 mm. In some examples, the standardized mesh or custom fitted mesh may have a specific size. The specific size of the standardized mesh or custom fitted mesh may be quantified as a cross-sectional area, where the “cross-sectional area” is the two-dimensional area of the largest areal surface of the standardized mesh or custom fitted mesh. In some examples, the cross-sectional area may range from: 25 cm² to 400 cm², 100 cm² to 400 cm², 225 cm² to 400 cm², 25 cm² to 225 cm², 25 cm² to 100 cm², or 225 cm² to 400 cm². In some examples, the standardized mesh or custom fitted mesh is a monofilament macroporous mesh.

Some examples of the method, system, device, and non-transitory computer readable medium further include implanting the custom fitted mesh into the patient. In some examples, the implanting is performed by at least one surgical robot. In some examples, the implanting is automated by the at least one surgical robot. For instance, the at least one surgical robot may, in certain implementations, automatically insert and place the custom fitted mesh in accordance with the topographical map. In some examples, the insertion and placement of the custom fitted mesh by the at least one surgical robot may be performed in accordance with instructions stored on a computer readable medium described herein. In certain examples, the insertion and placement of the custom fitted mesh by the at least one surgical robot may be performed with assistance from at least one machine learning algorithm. In some examples, the implanting is manually assisted by the at least one user operating the at least one surgical robot. In some such examples, the at least one user may be prompted to guide the custom fitted mesh toward the applicable portion of the patient's abdominal cavity using the at least one surgical robot. The at least one screen would then, in some embodiments, display the topographical map of the applicable portion of the patient's abdominal cavity (which may have been previously generated to form the custom fitted mesh.) the at least one user may then be prompted to align the custom fitted mesh with the topographical map, such as by displaying a real-time representation of the aligning on the at least one screen. In some examples, the implanting is performed manually by the at least one user. In some such examples, the at least one 3D printer may print instructions on the custom fitted mesh for implanting the custom fitted mesh into the patient. In certain examples, the printed instructions may specifically instruct the at least one user regarding how to insert and align the mesh to avoid slippage.

In certain implementations, the at least one user may be notified (e.g., visually on the at least one screen) when the custom fitted mesh has been installed and properly aligned—i.e., that the contours of the custom fitted mesh align to the contours of the relevant portion of the patient's abdominal cavity. In some examples, the at least one user may be notified if the custom fitted mesh slips out of place during installation, so that the at least one user has an opportunity to re-position the mesh before stitching up the patient. In some examples, the at least one user may be notified if the custom fitted mesh slips out of place after installation, so that corrective surgery can be performed.

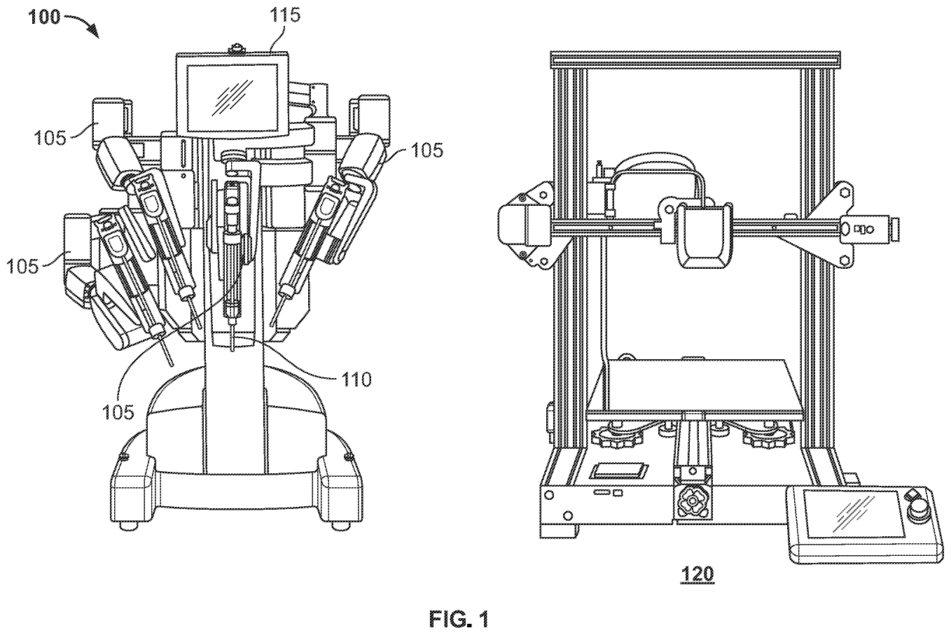

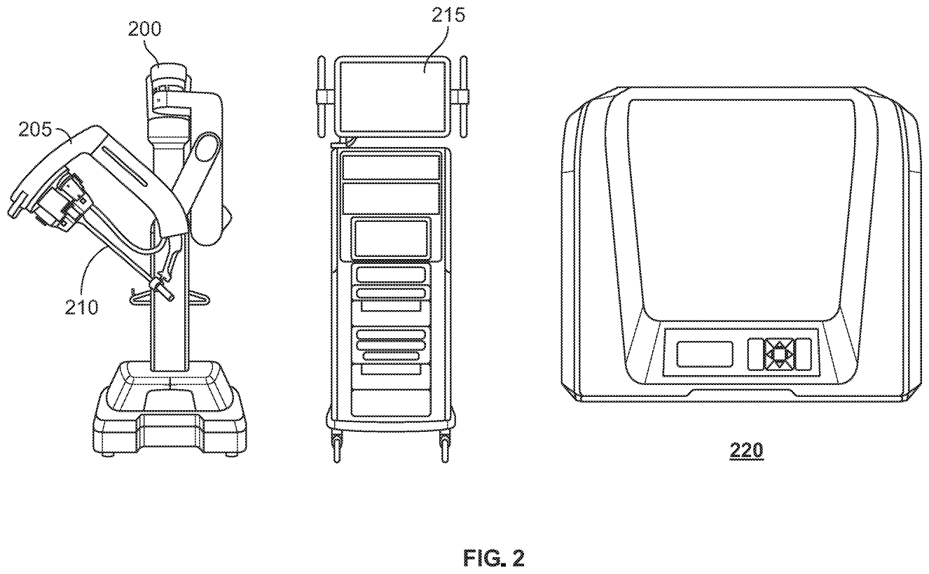

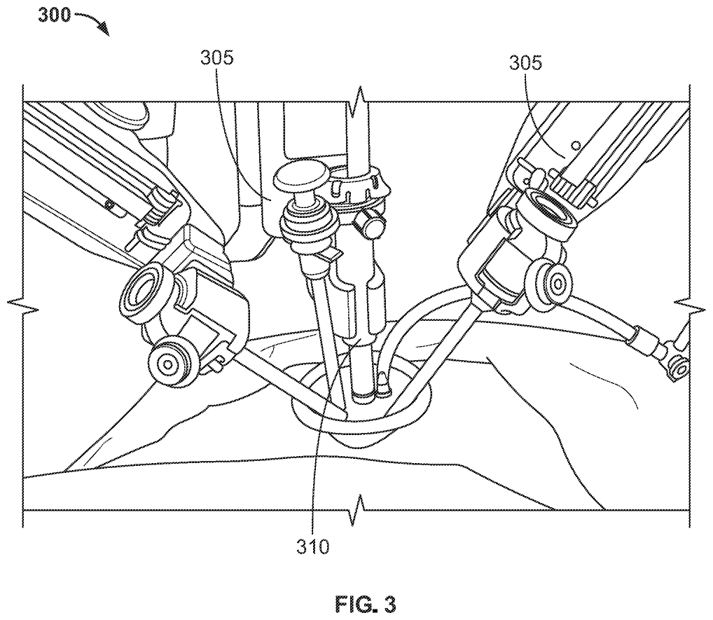

FIG. 1 shows an example of a system for forming a custom fitted mesh based on a topographical map of at least one portion of a patient's abdominal cavity according to aspects of the present disclosure. The example shown includes device 100, and machine 120. In some examples, the at least one device 100 is at least one surgical robot which is an example of, or includes aspects of, the surgical robot described with reference to FIGS. 2 and 3.

In one embodiment, device 100 includes at least one arm 105 and screen 115. At least one arm 105 is an example of, or includes aspects of, the corresponding element described with reference to FIGS. 2 and 3. In one embodiment, at least one arm 105 includes at least one camera 110. Camera 110 is an example of, or includes aspects of, the corresponding element described with reference to FIGS. 2 and 3. Screen 115 is an example of, or includes aspects of, the corresponding element described with reference to FIGS. 2, 4 and 5.

According to some embodiments, device 100 allows at least one user to obtain, using at least one camera 110 of at least one device 100, a real-time representation of at least one portion of a patient's abdominal cavity. In some examples, device 100 displays the real-time representation of the at least one portion of the patient's abdominal cavity on at least one screen 115. In some examples, device 100 allows the at least one user of the least one device 100 to create a topographical map of a contour of the at least one portion of the patient's abdominal cavity. In some examples, device 100 instructs at least one machine 120 to form a custom fitted mesh based on the topographical map, where the custom fitted mesh conforms to the contour of the at least one portion of the patient's abdominal cavity. In some examples, the at least one camera 110 is at least one surgical camera.

In some examples, the at least one machine 120 includes at least one 3D printer. In some examples, machine 120 forms the custom fitted mesh by 3D printing the custom fitted mesh based on the topographical map. In some examples, the at least one machine 120 includes a press machine 120. In some examples, machine 120 obtains a standardized mesh. In some examples, machine 120 presses, based on the topographical map, the standardized mesh into the custom fitted mesh.

In some embodiments, device 100 includes a computing device such as a personal computer, laptop computer, mainframe computer, palmtop computer, personal assistant, mobile device, or any other suitable processing device.

In some examples, a screen 115 may include, or be a part of, a display. A display may comprise a conventional monitor, a monitor coupled with an integrated display, an integrated display (e.g., an LCD or LED display), or other means for viewing associated data or processing information, such as but not limited to a screen of a virtual reality (VR) or augmented reality (AR) device. Output devices 100 other than the display can be used, such as printers, other computers or data storage devices 100, and computer networks. In some examples, the at least one screen 115 is part of the at least one device 100.

Device 100 may include an optical instrument (e.g., an image sensor, camera, etc.) for recording or capturing images, which may be stored locally, transmitted to another location, etc. For example, the at least one camera 110 may generally include at least one electromagnetic sensor. An electromagnetic sensor may capture visual information using one or more photosensitive elements that may be tuned for sensitivity to a spectrum of electromagnetic radiation. In some examples, the electromagnetic radiation may comprise visual light, infrared radiation, ultraviolet radiation, or a combination thereof. In certain examples where the at least one camera 110 is an imaging device as described above, the electromagnetic radiation may comprise ultrasonic radiation, x-rays, or any other suitable form of electromagnetic radiation. The resolution of visual information may be measured in pixels, where each pixel may relate an independent piece of captured information. In some cases, each pixel may thus correspond to one component of, for example, a two-dimensional (2D) Fourier transform of an image. Computation methods may use pixel information to reconstruct images captured by the device 100. In a camera, an image sensor may convert light incident on a camera lens into an analog or digital signal. An electronic device 100 may then display an image on a display panel based on the digital signal. Image sensors are commonly mounted on electronics such as smartphones, tablet personal computers (PCs), laptop PCs, and wearable devices.

In some examples, device 100 may include a processor. A processor is an intelligent hardware device, (e.g., a general-purpose processing component, a digital signal processor (DSP), a central processing unit (CPU), a graphics processing unit (GPU), a microcontroller, an application specific integrated circuit (ASIC), a field programmable gate array (FPGA), a programmable logic device, a discrete gate or transistor logic component, a discrete hardware component, or any combination thereof). In some cases, the processor is configured to operate a memory array using a memory controller. In other cases, a memory controller is integrated into the processor. In some cases, the processor is configured to execute computer-readable instructions stored in a memory to perform various functions. In some embodiments, a processor includes special purpose components for modem processing, baseband processing, digital signal processing, or transmission processing.

Software may include code to implement aspects of the present disclosure. Software may be stored in a non-transitory computer-readable medium such as system memory or other memory. In some cases, the software may not be directly executable by the processor but may cause a computer (e.g., when compiled and executed) to perform functions described herein.

According to some embodiments, a processor receives, from at least one camera 110 of at least one device 100, data corresponding to a real-time representation of at least one portion of a patient's abdominal cavity. In some examples, a processor instructs at least one screen 115 to display the real-time representation of the at least one portion of the patient's abdominal cavity. In some examples, a processor receives instructions from at least one user of the least one device 100 to create a topographical map of a contour of the at least one portion of the patient's abdominal cavity. In some examples, a processor instructs at least one machine 120 to form a custom fitted mesh based on the topographical map of the at least one portion of the patient's abdominal cavity, where the custom fitted mesh conforms to the contour of the at least one portion of the patient's abdominal cavity.

In some examples, device 100 may include memory. Examples of a memory device include random access memory (RAM), read-only memory (ROM), or a hard disk. Examples of memory devices include solid state memory and a hard disk drive. In some examples, memory is used to store computer-readable, computer-executable software including instructions that, when executed, cause a processor to perform various functions described herein. In some cases, the memory contains, among other things, a basic input/output system (BIOS) which controls basic hardware or software operation such as the interaction with peripheral components or devices. In some cases, a memory controller operates memory cells. For example, the memory controller can include a row decoder, column decoder, or both. In some cases, memory cells within a memory store information in the form of a logical state.

FIG. 2 shows an example of a system for forming a custom fitted mesh based on a topographical map of at least one portion of a patient's abdominal cavity according to aspects of the present disclosure. The example shown includes surgical robot 200, external screen 215, and 3D printer 220. Surgical robot 200 is an example of, or includes aspects of, the corresponding element described with reference to FIGS. 1 and 3. In one embodiment, surgical robot 200 includes at least one arm 205. At least one arm 205 is an example of, or includes aspects of, the corresponding element described with reference to FIGS. 1 and 3.

According to some embodiments, surgical robot 200 inserts the at least one surgical camera 210 into the patient using the at least one surgical robot 200. In some examples, surgical robot 200 uses the at least one surgical robot 200, navigating the at least one surgical camera 210 toward the at least one portion of the patient's abdominal cavity. In some examples, surgical robot 200 captures, using the at least one surgical camera 210, the real time representation of the at least one portion of the patient's abdominal cavity.

In one embodiment, at least one arm 205 includes surgical camera 210. In some examples, the at least one surgical camera 210 includes a laparoscopic camera. In some examples, the at least one surgical camera 210 includes a robotic camera, where the robotic camera is present on at least one surgical robot 200. Surgical camera 210 is an example of, or includes aspects of, the corresponding element described with reference to FIGS. 1 and 3.

In some examples, the at least one screen is at least one external screen 215. In some examples, the at least one external screen 215 is present on a computer, a mobile device, as a projected image, or any combination thereof.

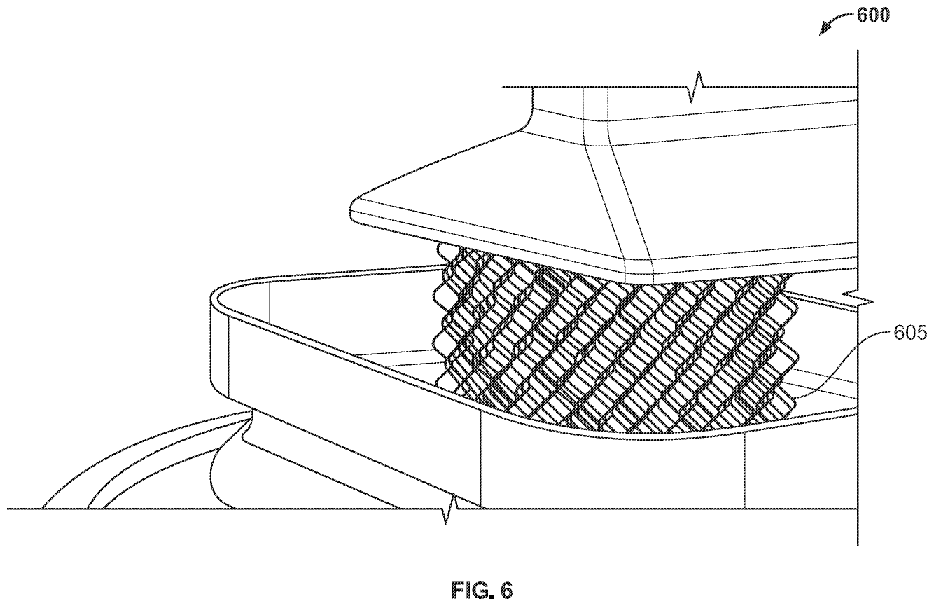

3D printer 220 is an example of, or includes aspects of, the corresponding element described with reference to FIG. 6.

FIG. 3 shows an example of a system for forming a custom fitted mesh based on a topographical map of at least one portion of a patient's abdominal cavity according to aspects of the present disclosure. Surgical robot 300 is an example of, or includes aspects of, the corresponding element described with reference to FIGS. 1 and 2. In one embodiment, surgical robot 300 includes at least one arm 305. At least one arm 305 is an example of, or includes aspects of, the corresponding element described with reference to FIGS. 1 and 2. In one embodiment, at least one arm 305 includes surgical camera 310. Surgical camera 310 is an example of, or includes aspects of, the corresponding element described with reference to FIGS. 1 and 2.

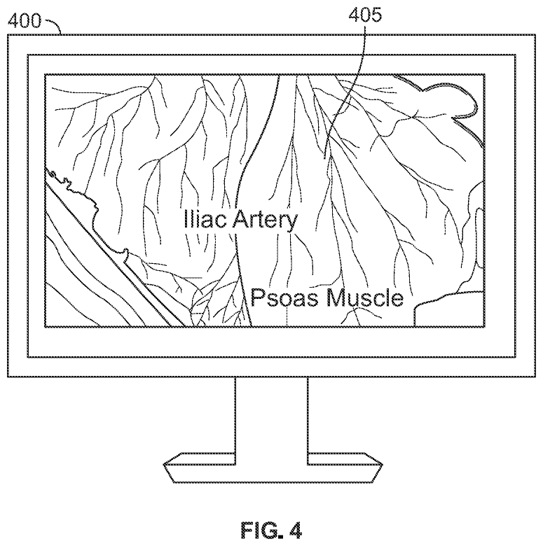

FIG. 4 shows an example of a real-time representation of at least one portion of a patient's abdominal cavity 405, as displayed on a screen 400 according to aspects of the present disclosure. Screen 400 is an example of, or includes aspects of, the corresponding element described with reference to FIGS. 1, 2, and 5.

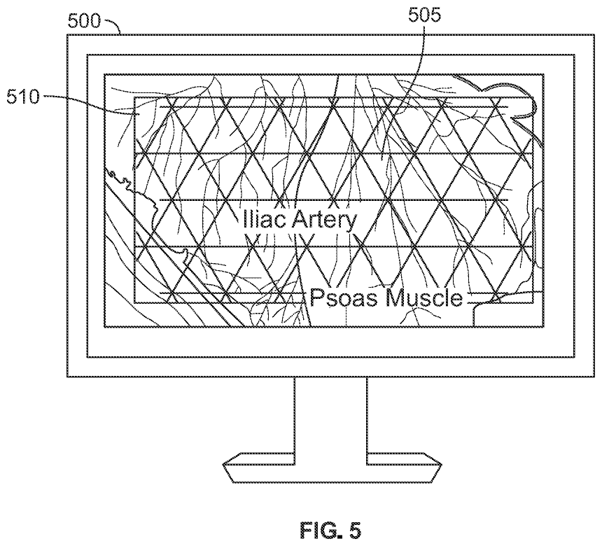

In one embodiment, screen 400 includes real-time representation of at least one portion of a patient's abdominal cavity 405. In some examples, the real-time representation of at least one portion of a patient's abdominal cavity 405 is a real time representation of the patient's myopectineal orifice and pelvic floor and where the contour of the at least one portion of the patient's abdominal cavity is a contour of the patient's myopectineal orifice and pelvic floor. Real-time representation of at least one portion of a patient's abdominal cavity 405 is an example of, or includes aspects of, the corresponding element described with reference to FIG. 5.

FIG. 5 shows an example of a topographical map of a contour on a real-time representation of at least one portion of a patient's abdominal cavity 505, as displayed on a screen 500 according to aspects of the present disclosure. The example shown includes screen 500. Screen 500 is an example of, or includes aspects of, the corresponding element described with reference to FIGS. 1, 2, and 4.

In one embodiment, screen 500 includes real-time representation of at least one portion of a patient's abdominal cavity 505. Real-time representation of at least one portion of a patient's abdominal cavity 505 is an example of, or includes aspects of, the corresponding element described with reference to FIG. 4. In one embodiment, real-time representation of at least one portion of a patient's abdominal cavity 505 includes topographical map of a contour of the at least one portion of the patient's abdominal cavity 510.

In some examples, the topographical map is virtually overlaid over the real-time representation of the at least one portion of the patient's abdominal cavity. In some examples, topographical map of a contour of the at least one portion of the patient's abdominal cavity 510 allows the at least one user to determine a location and dimensions of at least one hernia defect. In some examples, topographical map of a contour of the at least one portion of the patient's abdominal cavity 510 allows the at least one user to modify the topographical map of the at least one portion of the patient's abdominal cavity to omit the at least one hernia defect.

According to some embodiments, a surgical robot (e.g., a surgical robot described herein, for example, with reference to FIGS. 2 and 3) selects a pre-set topographical template using the at least one screen 500. In some examples, a surgical robot overlays, using the at least one screen 500, the pre-set topographical template over the real-time representation of the at least one portion of the patient's abdominal cavity. In some examples, a surgical robot warps the pre-set topographical template to match the contour of the at least one portion of the patient's abdominal cavity. In some examples, the warping is performed using the at least one screen 500. In some examples, the warping is performed by at least one user using the at least one camera. In some examples, a surgical robot 515 defines a fixed point on the real-time representation of at least one portion of a patient's abdominal cavity 505. In some examples, a surgical robot defines a set of end points on the real-time representation of at least one portion of a patient's abdominal cavity 505. In some examples, a surgical robot defines a set of vectors, where each of the set of vectors starts at the fixed point and terminates at one of the end points. In some examples, a surgical robot calculates the vector map, where the vector map is calculated by determining a vector space defined by the set of vectors. In some examples, a surgical robot displays the vector map on the at least one screen 500. In some examples, a surgical robot displays the vector map on at least one second screen 500. In some examples, a surgical robot implants the custom fitted mesh into the patient. In some examples, the implanting is manually assisted by the at least one user operating the at least one surgical robot. In some examples, the implanting is performed manually by the at least one user.

FIG. 6 shows an example of a 3D printer 600 used to form a custom fitted mesh 605 according to aspects of the present disclosure. The example shown includes 3D printer 600 and custom fitted mesh 605. 3D printer 600 is an example of, or includes aspects of, the corresponding element described with reference to FIG. 2.

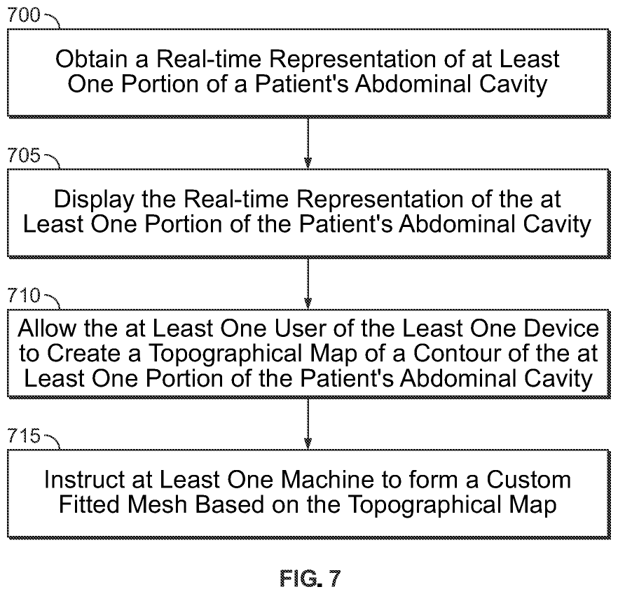

FIG. 7 shows an example of a process for forming a custom fitted mesh based on a topographical map of at least one portion of a patient's abdominal cavity according to aspects of the present disclosure. In some cases, the operations described herein are composed of various substeps, or are performed in conjunction with other operations. At operation 700, the system allows at least one user to obtain, using at least one camera of at least one device, a real-time representation of at least one portion of a patient's abdominal cavity. In some cases, the operations of this step refer to, or may be performed by, a device and camera as described with reference to FIG. 1. At operation 705, the system displays the real-time representation of the at least one portion of the patient's abdominal cavity on at least one screen. In some cases, the operations of this step refer to, or may be performed by, a device and screen as described with reference to FIG. 1. At operation 710, the system allows the at least one user of the least one device to create a topographical map of a contour of the at least one portion of the patient's abdominal cavity. In some cases, the operations of this step refer to, or may be performed by, a device and screen as described with reference to FIG. 1. At operation 715, the system instructs at least one machine to form a custom fitted mesh based on the topographical map, where the custom fitted mesh conforms to the contour of the at least one portion of the patient's abdominal cavity. In some cases, the operations of this step refer to, or may be performed by, a device as described with reference to FIG. 1.

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

The described systems and methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise random access memory (RAM), read-only memory (ROM), electrically erasable programmable read-only memory (EEPROM), compact disk (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

1. A method comprising:
allowing at least one user to obtain, using at least one camera of at least one device, a real-time representation of at least one portion of a patient's abdominal cavity;
displaying the real-time representation of the at least one portion of the patient's abdominal cavity on at least one screen;
allowing the at least one user of the least one device to create a topographical map of a contour of the at least one portion of the patient's abdominal cavity; and
instructing at least one machine to form a custom fitted mesh based on the topographical map, where the custom fitted mesh conforms to the contour of the at least one portion of the patient's abdominal cavity.

2. The method of claim 1, where the at least one camera is at least one surgical camera, where the at least one device is at least one surgical robot, and where obtaining the real-time representation comprises:
inserting the at least one surgical camera into the patient using the at least one surgical robot;
using the at least one surgical robot, navigating the at least one surgical camera toward the at least one portion of the patient's abdominal cavity; and
capturing, using the at least one surgical camera, the real time representation of the at least one portion of the patient's abdominal cavity.

3. The method of claim 1, where the at least one screen is part of the at least one device.

2. The method of claim 1, where the at least one screen is at least one external screen.

5. The method of claim 1, where the topographical map is virtually overlaid over the real-time representation of the at least one portion of the patient's abdominal cavity.

6. The method of claim 1, where the topographical map is created by:
selecting a pre-set topographical template using the at least one screen;
overlaying, using the at least one screen, the pre-set topographical template over the real-time representation of the at least one portion of the patient's abdominal cavity; and
warping the pre-set topographical template to match the contour of the at least one portion of the patient's abdominal cavity.

7. The method of claim 1, where the topographical map is a vector map, where the vector map is created by:
defining fixed point on the real-time representation of the at least one portion of the patient's abdominal cavity;
defining a plurality of end points on the real-time representation of the at least one portion of the patient's abdominal cavity;
defining a plurality of vectors, where the plurality of vectors starts at the fixed point and terminates at one of the end points; and
calculating the vector map, where the vector map is calculated by determining a vector space defined by the plurality of vectors.

8. The method of claim 1, where allowing at least one user of the at least one device to create the topographical map of the at least one portion of the patient's abdominal cavity comprises:
allowing the at least one user to determine a location and dimensions of at least one hernia defect; and
allowing the at least one user to modify the topographical map of the at least one portion of the patient's abdominal cavity to omit the at least one hernia defect.

9. The method of claim Error! Reference source not found., where forming the custom fitted mesh comprises 3D printing the custom fitted mesh based on the topographical map.

10. The method of claim 1, where forming the custom fitted mesh comprises:
obtaining a standardized mesh; and
pressing, based on the topographical map, the standardized mesh into the custom fitted mesh.

11. The method of claim 1, where the at least one portion of the patient's abdominal cavity was previously dissected.

12. The method of claim 1, where the custom fitted mesh is a monofilament macroporous mesh.

13. The method of claim 1, where the real-time representation of the at least one portion of the patient's abdominal cavity comprises a real time representation of the patient's myopectineal orifice and pelvic floor.

14. The method of claim 1, where the contour of the at least one portion of the patient's abdominal cavity comprises a contour of the patient's myopectineal orifice and pelvic floor.

15. A system comprising:
at least one device comprising:
at least one screen,
where the at least one screen is configured to display a real-time representation of at least one portion of a patient's abdominal cavity;
at least one camera,
where the at least one camera is configured to obtain a real-time representation of the at least one portion of the patient's abdominal cavity,
where the at least one camera is configured to allow at least one user of the at least one device to create a topographical map of the at least one portion of the patient's abdominal cavity from the real-time representation; and
at least one machine,
where the at least one machine is configured to form a custom fitted mesh based on the topographical map of the at least one portion of the patient's abdominal cavity.

16. The system of claim 15, where the at least one device is at least one surgical robot.

17. The system of claim 16, where the at least one camera is present on the at least one surgical robot.

38. The system of claim 15, where the at least one machine comprises a 3D printer.

194. The system of claim 15, where the at least one machine comprises a press machine.

20. A non-transitory computer readable medium storing code, the code comprising instructions executable by a processor to:
receive, from at least one camera of at least one device, data corresponding to a real-time representation of at least one portion of a patient's abdominal cavity;
instruct at least one screen to display the real-time representation of the at least one portion of the patient's abdominal cavity;
receive instructions from at least one user of the least one device to create a topographical map of a contour of the at least one portion of the patient's abdominal cavity; and
instruct at least one machine to form a custom fitted mesh based on the topographical map of the at least one portion of the patient's abdominal cavity, where the custom fitted mesh conforms to the contour of the at least one portion of the patient's abdominal cavity.
